(19) United States
(12) Patent Application Publication
(10) Pub. No.: US 2022/0156864 A1
(43) Pub. Date: May 19, 2022

(54) System and Method for Electronically Consenting to Engage in Sexual Activity

(71) Applicants: Elliot M. Furman, New York, NY (US); Jayson Scott Slotnik, Bethesda, MD (US)

(72) Inventors: Elliot M. Furman, New York, NY (US); Jayson Scott Slotnik, Bethesda, MD (US)

(21) Appl. No.: 17/098,367

(22) Filed: Nov. 14, 2020

(51) Int. Cl. G06Q 50/18; G06Q 10/10; G06Q 50/26; G06K 7/10; G06Q 30/00; H04L 9/32; H04W 4/029; H04W 4/80; H04W 4/90; A61F 6/00; A61F 6/04

(52) U.S. Cl. CPC G06Q 50/188; G06Q 10/10; G06Q 50/265; G06K 7/10861; G06Q 30/0185; G06K 2007/10504; H04W 4/029; H04W 4/80; H04W 4/90; A61F 6/005; A61F 6/04; H04L 9/3247

(57) ABSTRACT

A system and method for electronically consenting to engage in sexual activity enables two people, the consenters, to use their mobile communication devices to generate and execute a sexual consent contract, and to withdraw consent. The system and method verifies that the consenters would like to engage in the sexual activity and are in proximal communication and cooperating with each other. A sexual consent contract is electronically generated, which includes a consent period during which the sexual activity may take place. The contract is electronically signed by the consenters and a fully executed sexual consent contract is stored. The parties may withdraw their consent for a limited period of time after the contract is electronically signed. A withdrawal of consent by one person alerts the other person. Upon withdrawal, both people can provide notes as to the nature of their encounter. Those notes are stored with the fully executed sexual consent contract. The contract and additional information stored with it, such as notes, may be accessed by investigators in a legal or procedural proceeding for use as evidence to aid in the adjudication of any dispute as to the consensual nature of the sexual encounter.

### BACKGROUND

“Consent” means a voluntary and revocable agreement to participate in a sexual act. Consent expresses desire, not simply acquiescence. Consent is affirmative in that consent is a clear and freely given “yes”, not the absence of “no.”

Such affirmative consent is a knowing, voluntary, and mutual decision among all participants to engage in sexual activity. Consent can be given by words or actions, as long as those words or actions create clear permission regarding willingness to engage in the sexual activity. Silence or lack of resistance, in and of itself, does not demonstrate consent. The definition of consent does not vary based upon a participant's sex, sexual orientation, gender identity, or gender expression.

Sexual violence refers to physical sexual acts perpetrated against a person's will or where a person is incapable of giving consent due to, for example, the victim's use of drugs or alcohol. A number of different acts fall into the category of sexual violence, including rape, sexual assault, sexual battery, and sexual coercion. The terms “sexual violence” and “sexual assault” are used interchangeably herein.

Victims of alleged sexual assault often have options to pursue legal action through civil litigation or by pressing criminal charges. Furthermore, many educational institutions such as colleges and universities (“post-secondary institutions”) are bound by federal regulations called Title IX in responding to and adjudicating instances of alleged sexual assault. The government has used Title IX to compel schools to address sexual violence. Universities that fail to do so risk losing their federal funding.

Title IX does not replace civil or criminal justice systems. However, the way in which a school, college, or university responds to allegations of sexual harassment in an education program or activity has serious consequences for the complainant, for the respondent, and for the school.

In cases of alleged sexual assault, it has heretofore been difficult, if not impossible, to establish or prove that consent was given by either or both parties. Even if consent was given, it has heretofore been difficult or impossible to establish or prove that consent was revoked by either of the parties. Proving consent, withdrawal of consent, and the facts surrounding a sexual encounter and alleged sexual assault is critical for any criminal investigation and proceeding, civil ligation, and Title IX proceeding.

Thus, a need presently exists for a system and method for electronically consenting to engage in sexual activity.

### SUMMARY

A method for electronically consenting to engage in a sexual activity between a first person and second person. A first mobile communication device of a first person is provided. A second mobile communication device of a second person is provided. It is electronically verified that the first person and the second person would like to engage in sexual activity and are in proximal communication and cooperating with each other. A sexual consent contract is electronically generated for the first person and the second person. The sexual consent contract is displayed on the display of the first mobile communication device and on the display of the second mobile communication device.

The sexual consent contract is electronically signed, which includes receiving a first electronic signature of the first person from the first mobile communication device, and receiving a second electronic signature of the second person from the second mobile communication device. A fully executed sexual consent contract is created from the electronically signed sexual consent contract of the first person and the electronically signed sexual consent contract of the second person. The sexual consent contract comprises a consent period during which sexual activity may take place. The fully executed sexual consent contract is stored. It is determined if consent remained persistent for the first person and for the second person during the consent period.

### BRIEF DESCRIPTION OF THE DRAWINGS

FIG. 1 illustrates a method for electronically consenting to sexual behavior between a first person and a second person.

FIG. 2 shows a method of electronically verifying the intent of and proximity of the first person and the second person.

FIG. 3 shows a consent persistence determination method.

FIG. 4 shows an alternative embodiment of FIG. 1 which includes various optional safety checks.

FIG. 5 shows the system architecture of a sexual consent system.

FIG. 6 is a consent server.

FIG. 7 shows a condom wrapper comprising a machine-readable verification code being scanned by the mobile communication devices of a first person and a second person.

FIG. 8. shows a method of electronically verifying the intent and proximity of the first person and the second person with a product having a machine-readable verification code.

FIG. 9 shows the architecture of an exemplary mobile communication device.

FIG. 10 shows a first machine-readable verification code encoded with the name of a first person, the first person's mobile phone number, and a universally unique identifier.

FIG. 11 shows a second machine-readable verification code encoded with the name of a second person, the second person's mobile phone number, and a universally unique identifier.

FIG. 12 shows a machine-readable verification encoded with a product identification code.

### DETAILED DESCRIPTION

Two people, Person 1 and Person 2, would like to engage in sexual activity with each other. The sexual activity may comprise any type of sexual conduct or activity such as, by way of example, kissing, suggestive touching, touching of genitalia, mutual masturbation, oral sex, vaginal sex, anal sex, and may also include acts that are not per se sexual but that some people engage in during sexual encounters because they find them pleasurable, exciting, erotic, or romantic such as choking, slapping, hitting, spitting, hair pulling, bondage, and any other type of erotic practices and roleplaying such as S&M (Sadism and Masochism) and BDSM (Bondage and Discipline, Dominance and Submission, and Sadism and Masochism). As is appreciated by those skilled in the art, there are nearly an endless multiplicity of sexual and erotic behaviors that people engage in. Disclosed herein are methods and systems for electronically consenting to any sexual or erotic encounter between two or more people. Furthermore, the methods and systems herein include an electronic legally enforceable means by which one or both people can prove consent was given or revoked.

Person 1 is also referred to herein as the First Person or User 1. Person 2 is also referred to herein as the Second Person or User 2. Person 1 and Person 2 each have a mobile communication device, herein referred to as mobile communication device 1 and mobile communication device 2, respectively. Mobile communication device 1 is also equivalently referred to herein as the first mobile communication device. Mobile communication device 2 is also equivalently referred to herein as the second mobile communication device.

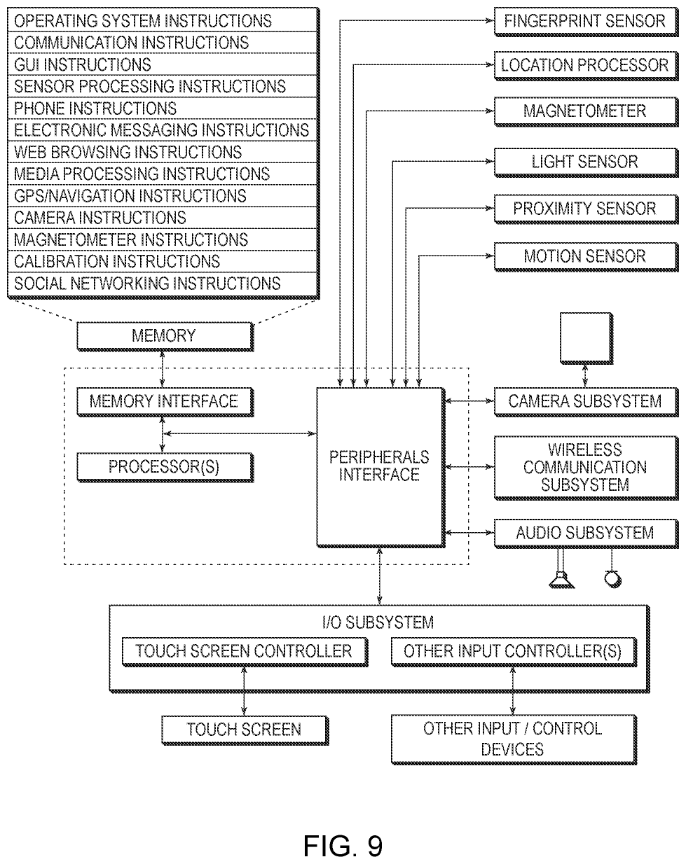

One example of a mobile communication device is a smartphone such as an iPhone or Android phone. Another example of a mobile computing device is a tablet such as a computer tablet such as an iPad, Samsung Galaxy, Microsoft Surface. Other types of mobile communication devices include smart watches and smart glasses. Those skilled in the art will appreciate that there are many types of mobile communication devices compatible with the present invention. FIG. 9 shows the architecture of an exemplary mobile communication device.

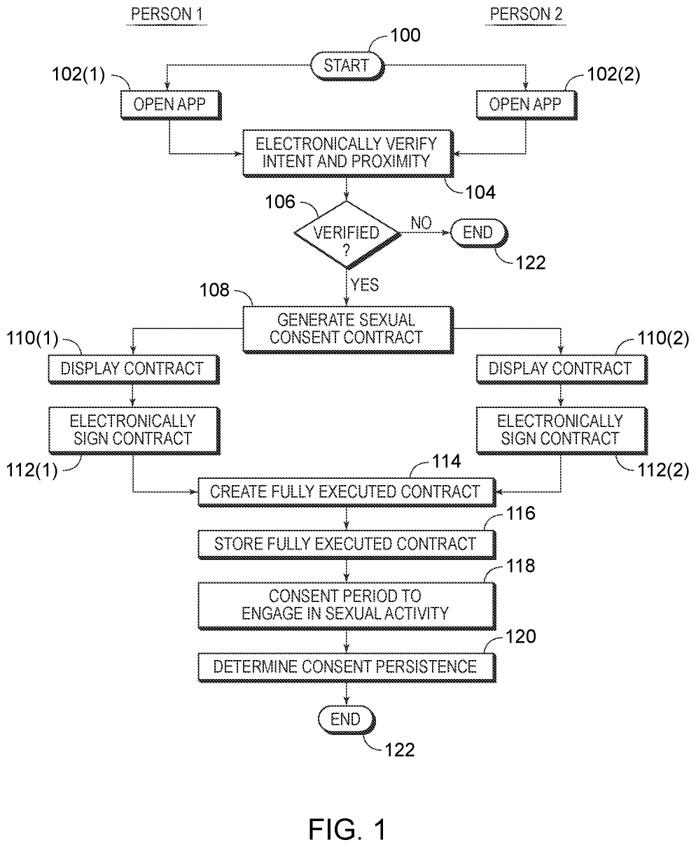

FIG. 1 illustrates a method for electronically consenting to sexual behavior between a first person and a second person. As will become clear, certain steps of the method of FIG. 1 operate as software running on the mobile communication device of each person's device (referred to herein as the “App”).

While this method, and other alternate methods disclosed herein, is shown for two people, it can may include three or more people, such as in an erotic, romantic, or sexual encounter between three people, commonly referred to as a ménage à trois, or in a group sex encounter where there are typically four or more people, generally referred to in the art, in various forms, as group sex, an orgy, a sex party, a gang bang, or any other sexual encounter involving many people. In the case of three or more people, the various steps shown and described, are repeated by and for the additional people, and between all of the people, as applicable.

Turning to FIG. 1, the method starts (100) with two people, Person 1 and Person 2, who ostensibly are sexually attracted to each other and would like to engage in some romantic, sexual, or erotic way. Both people, however, would like to affirmatively consent to any engagement, and to do so in a quick and unobtrusive, and legally binding manner. Furthermore, they would like to be able to revoke their consent at any time during the encounter or make a claim after the encounter that consent was withdrawn, and maintain a legal record in the case consent was revoked for any reason, including in the event the initially consensual encounter turned into a sexual assault, or in the case of differing recollections or opinions about the encounter after the fact. The words “revoke” and “withdraw” are used interchangeably herein with reference to consent. For example, “withdraw consent” and “revoke consent” shall be understood to have the same meaning.

As will be disclosed in greater detail later, the methods disclosed herein are stored as computer executable instructions in the memory of a mobile communication device as an application, or “app.” The instructions of the app when executed by the processor of the mobile communication device causes the mobile communication device to carry out the methods disclosed herein. Additionally, as will be disclosed in greater detail later, some steps of the methods may be stored as computer executable instructions in the memory of a computer server, which upon execution by the processor of the computer causes the computer to carry out the methods disclosed herein.

At some point prior to or proximate to any engagement, Person 1 and Person 2 open the app (step 102(1) for Person 1 and 102(2) for Person 2) on their mobile communication device. The timing of when the apps are opened can vary. For example, it could be done at a party or social gathering before the parties leave, it could be done during a date while out at dinner, or it could be done in the “heat of the moment” just before any sexual activity.

Verification

Next, the method electronically verifies that the First Person and the Second Person would like to engage in sexual activity with each other and are in proximal communication and cooperating with each other (104, 106). Namely, at steps 104 and 106, each app electronically verifies the intent of the First Person and the Second Person to engage sexually with each other, and that they are in proximal communication and interacting with each other. The verification process will be disclosed in detail below. These steps ensure that the parties (Person 1 and Person 2) are actually near each other, interacting, and cooperating with each other, such as in face-to-face communication with each other.

An important element of affirmative consent is that the parties are making a mutual decision prior to and proximate to any sexual activity, not remotely from each other, and not, for example, for some date far in the future. The consent is for a period of time close to when consent is granted. The consent is made in-person, not remotely. It is not an open-ended, never-ending form of consent. The parties therefore should be interacting and cooperating with each other, which is exactly what one or more of the disclosed steps accomplishes, as will disclosed in greater detail.

At step 106, if the step of verification 104 is not successful, the process ends 122. If the step of verification 104 is successful (“yes” branch of 106), the process continues to step 108. That is, after electronically verifying (step 104, 106) that the first person and the second person would like to engage in sexual activity and are in proximal communication and cooperating with each other, the method continues to step 108.

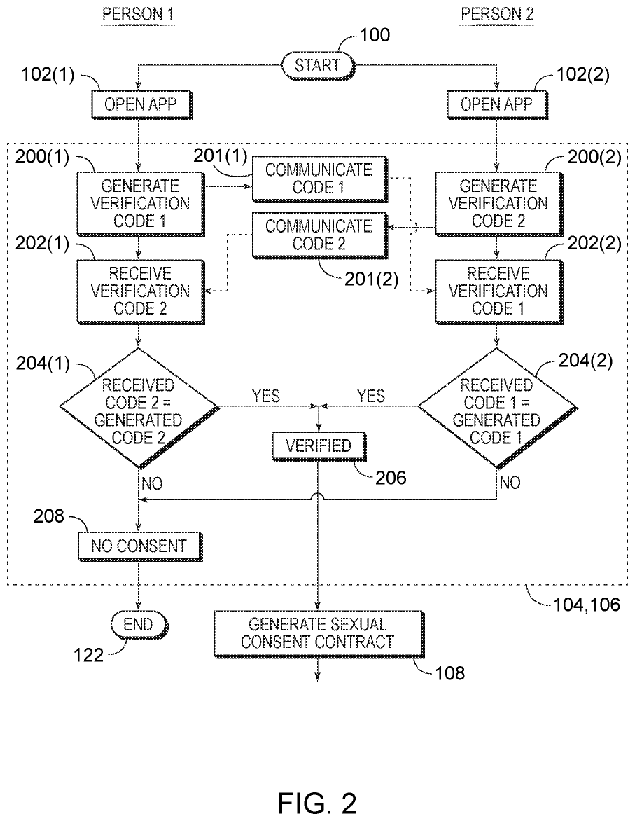

FIG. 2 shows a method of electronically verifying the intent of and proximity of the first person and the second person. By “intent” it is meant that the first person and second person would like to or have some mutual interest to engage in sexual activity with each other. The steps of electronically verifying (104, 106 of FIG. 1) are shown in expanded form within the dotted lines of box 104, 106 of FIG. 2. At step 200(1), a first verification code is generated for the first mobile communication device. At step 200(2), second verification code is generated for the second mobile communication device.

One example of a verification code is a randomly generated multidigit number, such as a 6 digit numerical code. This is just one example. There are however many other types of codes which may comprise multiple types of information such as, geolocation data, product information data, names, phone numbers, a time based one time password, a geolocation based one time password, a cryptographic hash. There are many other type of verification codes than can contain information like this.

Another type of verification code comprises a Universally Unique Identifier (“UUID”), for example in the form of a number represented in hexadecimal notation. The UUID has a length of several characters wherein each character is a hexadecimal number. One exemplary UUID comprises properties of the mobile communication device, such as MAC address (Media Access Controller identifier), phone number, International Mobile Equipment Identity (“IMEI”), and so forth. The UUID may comprise timestamps and geolocation information. The UUID may comprise identifying information such as name, address, biometric identifiers, and other information unique to a user of mobile communication device. The UUID may comprise product information, such as a product identification code (productID), company name, product name, lot number, manufacturer ID, expiration date, and the like. The UUID may comprise a cryptographic hash. A cryptographic hash function may take these and other data as a credentialed input to produce a fixed-size output of enciphered text as the hash value.

The UUID may comprise a random or pseudorandom number. In these ways, the verification code may be unique to each mobile communication device and identify the user of the mobile communication device.

One type of verification code is optically readable. A verification code may be optically readable by a human such as a numeric or alphanumeric code. This is an example of a human-readable code. A verification code may be machine readable by optical means, such as an optical barcode, such as a machine readable code. Types of machine-readable codes comprise an optical barcode, such as a barcode, a two-dimensional barcode (2D code), a matrix barcode like a QR code, and the like. Other type of verification code comprises contactless electronically readable codes such as a machine-readable optical code, an electromagnetic readable tag, and an electromagnetic readable-writable tag.

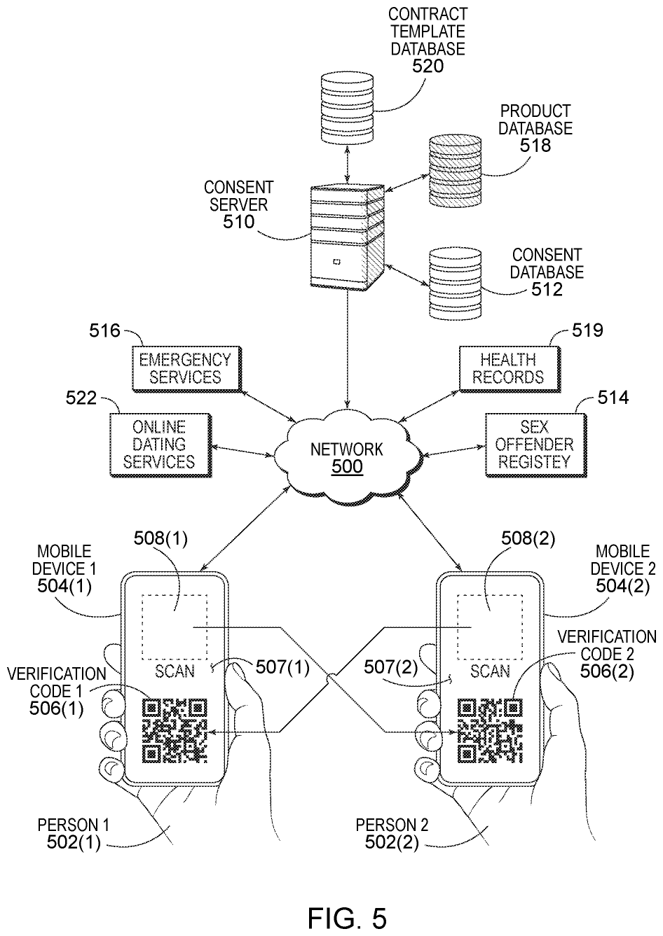

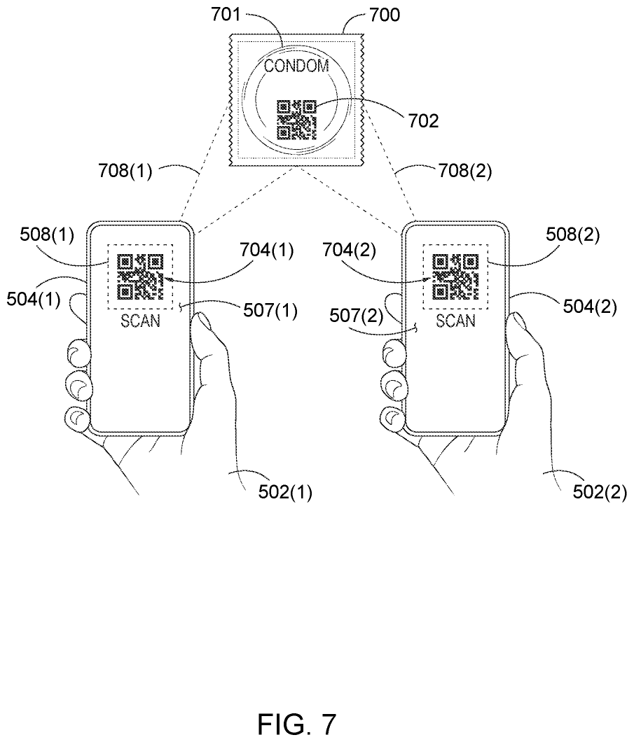

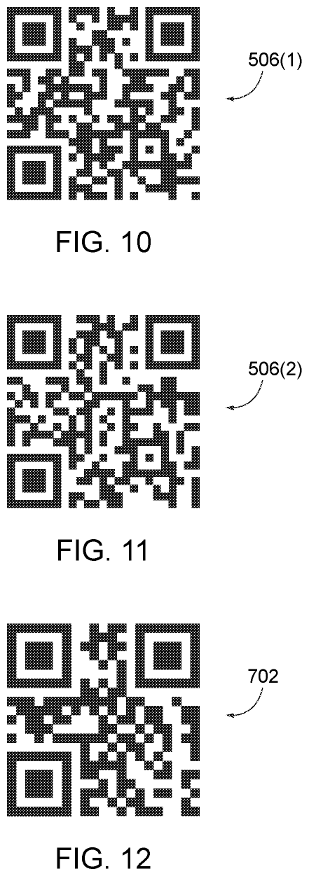

Two exemplary machine-readable verification codes are 506(1) and 506(2) of FIG. 5. FIGS. 10 and 11 show enlarged views of the machine-readable verification codes 506(1) and 506(2), respectively. These are QR codes are optically readable by a mobile communication device. These exemplary verification codes are encoded with data with data according having the format: FirstName;LastName;PhoneNumber;UUID. For example, when scanned by a mobile communication device, 506(1) decodes to: John;Doe;5553127482;a6b946. John Doe is name of Person 1 502(1), 5553127482 is his phone number, and a6b946 is the UUID. This exemplary UUID comprises 6 hexadecimal characters. This is just one exemplary UUID. A UUID may comprise more than or less than 6 hexadecimal characters. Similarly, when scanned by a mobile communication device, 506(2) decodes to: Jane;Doe;5558298823;9ad1b9. Jane Doe is the name of Person 2 502(2), 5558298823 is her phone number, and 9ad1b9 is the UUID. This is just one exemplary UUID. Another exemplary machine-readable verification code is 702 of FIG. 7. FIG. 12 shows an enlarged view of the machine-readable verification code 702. This optically machine-readable verification code has the format: ProductID. For example, when scanned by a mobile communication device, 702 decodes to: 7f27e8679938. This exemplary product identification code comprises 12 hexadecimal characters. This is just one exemplary product identification code. A product identification code may comprise more or less than 12 characters.

Turning back to FIG. 2, the verification codes are communicated at step 201. At step 201(1), the first verification code is communicated to the second mobile communication device. At step 202(2), the second verification code is communicated to the first mobile communication device.

In the case of an optically readable code, the first code is displayed on the display of the first mobile communication device at step 201(1), and the second code is displayed on the display of the second mobile communication device at step 201(2). For example, if the first code and second code are five-digit numbers, those number are displayed on the screen of the respective devices. In another embodiment, if the first and second codes are 2D barcodes, like a QR code, those barcodes are displayed on the screen of their respective devices.

Next, at step 202(1), the second verification code is received by the first mobile communication device. Similarly, at step 202(2), the first verification code is received by the second mobile communication device.

In the example above, wherein the code is a human-readable code, such as a five-digit numeric code, the second verification code is received on the first mobile communication device at step 202(1) by Person 1 who enters the second verification code on their mobile communication device, for example by typing the numbers of the code on the screen of the first mobile communication device. Similarly, the first verification code is received on the second mobile communication device at step 202(2) by the Person 2 who enters the first verification code on their mobile communication device, for example by typing the numbers of the code on the screen of the second mobile communication device.

In order for Person 1 to be able to enter the second verification code on their mobile communication device (the first mobile communication device) and Person 2 to be able to enter the first verification code on the mobile communication device (the second mobile communication device), Person 1 and Person 2 are necessarily interacting with each other, such as in face-to-face communication. In this example Person 2 would show their screen displaying the second verification code to the first person who then, upon reading it, enters it into their mobile communication device. Similarly, Person 1 would show their screen displaying the first varication code to the second person who then, upon reading it, enters it into their mobile communication device. Alternatively, Person 1 and Person 2 could read aloud the verification codes displayed on their screens to the other person. Alternatively, each mobile communication device can be configured to speak the verification codes over the speakers of their mobile communication devices by way of a text-to-speech software.

Various radio-based techniques implemented on a mobile communication device may be used to further verify that Person 1 and Person 2 are proximal to each other. For example, geolocation by way of the global positioning radio (GPS) may. In another example, WiFi or Bluetooth may be employed. The Bluetooth radio can be used to additionally verify that Person 1 and Person 2 are near each other. For example, when electronically verifying, the app can turn on the Bluetooth radio in each mobile communication device, if it is not already on. Bluetooth LE (“BLE”) may be employed. In one Bluetooth-based proximity-detection scheme, the mobile communication devices constantly pings out a signal. If the two phones are within range when the app is open and for a period of time, for example a minute, those two phones exchange unique verification codes, such as rotating identifier beacon numbers. The RSSI (Received Signal Strength Indicator) of each Bluetooth signal may be used to estimate the distance between devices. Bluetooth 5.1 supports Radio Direction Finding (“RDF”) and therefore is accurate to within approximately 1 cm. These codes are verifiable by each mobile communication device. In one embodiment the codes are generated by a consent server, which will be disclosed below, and may include each device type, MAC address (Media Access Controller identifier), phone number, International Mobile Equipment Identity (“IMEI”), and other properties of the mobile communication device. In this way, the verification code comprises a Universally Unique Identifier (“UUID”), for example in the form of a 128-bit number represented in hexadecimal notation.

In one embodiment verification code comprising the UUID is generated by the apps on each mobile communication device in communication with a consent server. For example, properties of each mobile communication device (or any other elements comprising the UUID) may be transmitted to the consent server, or retrieved from the consent server if already stored. The consent server may then generate the verification code, for example a 2D barcode. That barcode may be transmitted to the mobile communication device. When another mobile communication device scans that barcode as described herein, that scan is transmitted to the consent server. The scan is compared with the generated barcode to determine if they match.

In this embodiment where the first and second codes are 2D barcodes, such as a QR code, the second verification code is received on the first mobile communication device at step 202(1) by scanning the QR code displayed on the second mobile communication device with the first mobile communication device. Similarly, the first verification code received on the second mobile communication device at step 202(2) by scanning the QR code displayed on the first mobile communication device with the second mobile communication device. It is well understood by those having ordinary skill in the art how to configure a mobile communication device to scan a barcode such as a QR code by way of the camera of the mobile communication device.

In order for Person 1 to use the first mobile communication device to optically scan the barcode displayed on the second mobile communication device, and for Person 2 to use the second mobile communication device to scan the barcode displayed on the first mobile communication device, Person 1 and Person 2 must be in close proximal communication with each other and must interact with each other. For example, each person must place their phones in the correct orientation within inches of one another.

As an extra measure of verification security, short range wireless communication such as Bluetooth, Near Field Communication (NFC), or equivalent in each mobile communication device may be used to further establish that Person 1 and person 2 are close to each other. In this way, the mobile communication devices must be close enough for short range wireless to be effective. Some of these methods were disclosed above.

Furthermore, as an extra measure of verification security the method may time out, then there is accordingly no consent (step 208) and the method ends (steps 122) if the verification codes are not received (step 202(1) and 202(2)) within a preset period of time, for example within 90 seconds. In this way, the users of the mobile communication devices must be close to and interacting with each other to expeditiously communicate the verification codes to each other and scan or enter them.

As yet another extra measure of verification security, when the code is received in step 204(1) a first received code timestamp is stored, and when the code is received in step 204(2) a second received code timestamp is stored. Then, the difference between the first received code timestamp and the second received code timestamp is calculated. If the difference is greater than a period of time, for example, 90 seconds, then there is no consent (step 208) and the method ends (step 122). In this way, the users of the mobile communication devices must interreact and collaborate to quickly scan or enter the verification codes.

Turning back to FIG. 2, at step 204(1) the code the was received on the first mobile communication device (step 202(1)) is compared to the code that was generated (step 200(2)) for the second mobile communication device. Similarly, at step 204(2) the code that was received on the second mobile communication device (step 202(2)) is compared to the code that was generated (step 200(1)) for the first mobile communication device. If the code received by the first mobile communication device matches the code generated for the second mobile communication device, and the code received by the second mobile communication device matches the code generated for the first mobile communication, then it is electronically verified that the first person and the second person would like to engage in sexual activity with each other, also referred to herein as collaborate with each other, and are in proximal communication with each other, as indicated in step 206.

If either of the received codes do not match the required generated codes, then there is no electronic verification that the first person and the second person would like to collaborate and are in proximal communication with each other. Accordingly, there can be no electronic consent as shown in step 208, and the process ends at step 122.

In the case where there is electronic verification 206, the method continues as disclosed above to generate a sexual consent contract in step 108 of FIG. 1.

Contract and Electronic Signatures

At step 108, a sexual consent contract (the “Contract”, “Consent Agreement”,

“Sexual Consent Contract”, “Consent Contract”, “Sexual Consent Agreement”, or “Agreement”) is electronically generated. One exemplary sexual consent contract is shown in Exhibit A, “Sexual Consent Agreement”. The Contract is a bilateral contract, satisfied by mutual promises of all of the parties involved. The contract includes, the contract date (variable {Contract Date}), the parties involved, that is, the name of the

First Person (“Consenter 1”, variable {Consenter 1}) and the name of the Second Person (“Consenter 2”, variable {Consenter 2}); the provisions mutually agreed to by the parties, namely, an agreement that they consent to engage in sexual activity for a period of time called the Consent Period and defined by variable {Consent Period Start(date, time)} and {Consent Duration}; and an electronic signature section comprising electronic signatures {eSig 1} and {eSig 2} and corresponding timestamps {eSig 1 DateTime} and {eSig 2 DateTime}. {eSig 1} is the electronic signature of {Consenter 1} and {eSign 2} is the electronic signature of {Consenter 2}

The names or other identifying information of the parties may be automatically obtained during the step of electronically verifying 104 and electronically inserted into the variable fields {Consenter 1} and {Consenter 2}. Those field may, in some embodiments, be entered manually. In one embodiment, this identifying information may automatically obtained by the app from the user's mobile communication device, which stores the contact information of the owner. In another embodiment, this identifying information is embedded in one or more bar codes that are generated, displayed, and scanned by the mobile communication devices. The generation and scanning of these codes will be disclosed later.

The signature section includes fields to accept an electronic signature for each party, denoted {eSig 1} and {eSig 2}. The signature section further comprises a date time stamp that is automatically generated upon acceptance of the corresponding electronic signature, denoted by variables {eSig 1 DateTime} and {eSig 2 DateTime}.

After the Contract is electronically generated 108, it is displayed on the display of the first mobile communication device at step 110(1), and on the display of the second mobile communication at step 110(2). Person 1 and Person 2 can then independently and privately review the contract on their mobile device.

Next the parties each electronically sign the sexual consent contract displayed on their respective mobile communication device. At step 112(1) the First Person electronically signs the sexual consent contract which includes receiving a first electronic signature of the person {eSig1} from the first mobile communication device. The date and time of the receipt of {eSig1} is also stored in the timestamp field {eSig1 DateTime}. Similarly, at step 112(2) the Second Person electronically signs the sexual consent contract which includes receiving a second electronic signature of the second person {eSig2} from the second mobile communication device. The date and time of the receipt of {eSig2} is also stored in the field {eSig2 DateTime}. The electronic signatures mean that the first person and the second person agree to terms and condition of the sexual consent contract.

An electronic signature comprises a computer data compilation of any symbol or series of symbols or information executed, adopted, or authorized by an individual to be the legally binding equivalent of the individual's handwritten signature. A handwritten electronic signature may be applied to an electronic device by drawing on the screen of the mobile communication device with a finger or stylus. An electronic signature may comprise biometric identifiers. Biometrics include a method of verifying an individual's identity based on measurement of the individual's physical feature(s) or repeatable action(s) where those features and/or actions are both unique to that individual and measurable. (21 C.F.R. 11.3(b)(3) which is hereby incorporated by reference). Furthermore, biometrics include voiceprints, fingerprints, handprints, face scans, and retinal scans which are examples of biometric means of identifying an individual. A digital signature comprises an electronic signature based upon cryptographic methods of originator authentication, computed by using a set of rules and a set of parameters such that the identity of the signer and the integrity of the data can be verified.

In one embodiment the electronic signature comprises a fingerprint which is received by a fingerprint scanner of the mobile communication device. Other electronic signatures may be used, alone and in combination, including, a typed name, typed initials, a checkbox, a fingerprint, initials drawn on the screen of the mobile communication device, signatures drawn on the screen of the mobile communication device, a fingerprint match, a face identification match, an audio recording of a voice, a voice identification match, a video recording, and a retinal identification scan or photo of the eye taken with the camera of the mobile communication device. In one embodiment, the electronic signature comprises an audio recording of the consenter saying “I consent.” In another embodiment, the electronic signature comprises a video recording of the consenter saying “I consent.”

The method may include a signatures timeout period, for example, requiring that both parties electronically sign the Contract within a period of time from when the Contract is displayed on the mobile communication device, or within a period of time from the date of the contract {Contract Date} which includes a date and timestamp of the generation of the contract. One example of a period of time is, within two minutes of {Contract Date}. If the signatures of all parties are not received with that time, the process ends 122 and there is no consent. Alternatively, the app may display a “Cancel” button near the signature field so that either party can explicitly end the process 122, thereby not consenting to the engage with each other.

The method may also compare a received electronic signature with a stored reference electronic signature. In this embodiment, the received electronic signature must match the reference electronic signature for the method to continue. If they do not, the process ends 122. The reference electronic signature may be obtained and stored during a setup process of the App. The stored signature may be stored and the received signature compared to the stored signature on the user's mobile communication device or at the consent server.

Once all electronic signatures are received (step 112(1), 112(2)), at step 114 a fully executed sexual consent contract is created. The fully executed sexual consent contract includes the contract that was generated in step 108, including the contract date {Contract Date}, the names of the consenters {Consenter 1} and {Consenter 2}, the Consent Period defined by {Consent Period Start{(date, time)} and {Consent Duration}, the first electronic signature received in step 112(1) {eSig1} and timestamp {eSig1 DateTime}, the second electronic signature received in step 112(2) {eSig2} and timestamp {eSig2 DateTime}.

Next, at step 116, the fully executed sexual consent contract is stored. As will be discussed later, in one embodiment the fully executed sexual consent contract, which comprises the bilateral agreements signed by each party, is stored is a consent database. The fully sexual consent contract is stored securely. It may be encrypted. The fully sexual consent contract may be stored in a blockchain. The fully executed sexual consent contract may be stored using best practices or standards recommended or suggested for, or in manner that facilitates or simplifies or enhances electronic discover (eDiscovery) of the fully executed sexual consent contract. Electronic discovery, or eDiscovery, is the process of identifying and delivering electronic information that can be used as evidence in legal cases. Other information may be associated with and stored with the fully executed sexual consent contract, in the same manner in which the fully executed sexual consent contract is stored. Other information may include metadata. In another embodiment, the fully executed contract may be stored on each mobile communication device by the App.

The fully executed sexual consent contract, and additional information associated with, appended to, and stored with it such as withdrawal information and geolocation data, as will be disclosed below, may be retrieved by investigators in a legal or procedural proceeding for use as evidence to aid in the adjudication of any dispute as to the consensual nature of the sexual encounter.

The contract shown in Exhibit A is just one exemplary contract. The contract may comprises other elements, sections, or provisions to solidify the enforceability of the Contract under State and Federal laws such as, by way of example, what is being offered and accepted, that the parties are competent and making the agreement of free will, the mutuality of their obligation, that is, that the parties have a common intention, the legality of the contract and how it may be terminated, that there is a “meeting of the minds”, and so forth.

For example, the contract may comprise a section comprising a list of sexual activities that the consenters mutually agree to. When the contract is displayed to the consenters, as will disclosed below, the consenters can mutually agree to the activities, for example, by checking check boxes on the display of their mobile device, and/or by agreeing to certain items with initials or biometric identifiers.

In another example, the contract my comprises a section where one or both consenters warrant that they are currently using contraception on an ongoing or will use a contraceptive device their sexual encounter.

The contract may further comprise a ratchet clause. One example of a ratchet clause states that during an encounter, sexual activity may escalate in excess of what was previously consented to. The ratchet clause offers the consenters various options of how to treat such escalation, for example by requiring mutual verbal consent, or by requiring retroactive checking of the activities in the activity section described above, or by explicitly agreement that there shall be no sexual activity of any kind other than what is specified in the optional activity section described above.

The contract may also comprise a section concerning accidental violations. One type of accidental violation is when one party accidentally violates the other, for example if a male through no fault or without intent penetrates a female. In this section the parties can agree whether such an incident shall be considered an assault or an accident, and where the burden of proof lies.

The contract may additionally comprise a non-disclosure agreement. The contract may comprise an agreement not to record the encounter with audio or video.

The contract may comprise a failure to perform clause. A failure to perform clause states that while the parties agree to participate in sexual activities, there is no guarantee that that they will have the ability to perform those activities. The clause may state that any failure to perform shall not be considered a violation of the Agreement, and that the consenters waive any right to legal redress for failure to perform.

The contract may comprise a witness section including a witness' electronic signature and date.

This is just one example of a contract and several examples of contract elements. Other elements and wording may be recommended or necessary according to the locality in which the contract is entered into, and the locality in which it may be interpreted and/or litigated. This will be disclosed in greater detail below.

With this disclosure, those having ordinary skill in the art of contract law will appreciate that the contract may comprise other and alternative elements, clauses, and sections, and that the contract may be worded and structured in various ways.

Consent Period

Step 118 is a Consent Period. As disclosed above, the Consent Period is the period of time during which the parties may engage in sexual activity, or “hook up” as some people refer to it, in accordance with the fully executed sexual consent contract of step 116. That is, during the Consent Period, the sexual consent contract is in-force in that the parties mutually agreed that they may engage in consensual sexual activity during that time.

Variables {Consent Period Start(date, time)} and {Consent Duration} define the Consent Period. {Consent Period Start(date, time)}comprises a date and time. In various embodiments, {Consent Period Start(date, time)} may be the date and time of generation of the sexual consent contract in step 108, or the later of the electronic signature time stamps, that is either {eSig1 DateTime} or {eSig2 DateTime}. Variable {Consent Duration} sets the Consent Period which begins at {Consent Period Start(date, time)}.

In one embodiment, the Consent Period is fixed. One example of fixed period of time is two (2) hours, for example {Consent Duration}=2. In another embodiment the Consent Time Period is variable and set by one or both people via their Apps prior to signing (steps 112(1) and 112(2)), that is {Consent Duration} is variable and in various embodiments set as mutually agreed to by the parties.

In another embodiment, the method defaults to a fixed period of time, however each person may change their desired time and the method selects the shortest of either the fixed period, or of the desired time entered by one or both people.

The method may display the Consent Period (the value or proposed value of {Consent Duration}) on the display of each user's mobile communication device. In one embodiment, the Consent Period is displayed on the display of each user's mobile communication device and each user must confirm that period, thereby mutually agreeing to the Consent Period.

Furthermore, the method may display a countdown timer counting down the time before the Consent Period expires. The method may also include an alarm that goes off on each user's mobile communication device upon or around the expiration of the Consent Period. For example, the alarm may be, alone and in combination, visual, audible, or haptic, such as vibratory.

Additionally, the method may display a button on each of the user's mobile telecommunication device to extend the Consent Period. For example, a button may be displayed to extend the Consent Period in fifteen-minute increments. In one example, both users must consent to extending the Consent Period. For example, if Person 1 extends the Consent Period by 30 minutes, a request to extend the Consent Period by that amount of time is displayed on the second mobile communication device. If Person 2 consents to the extension request, the Consent Contract is amended with a new Consent Period, namely {Consent Duration 2} and the new Consent Period replaces the old consent period {Consent Duration}. The Consent Period may further be extended with {Consent Duration 3} and so forth.

As will be disclosed in greater detail, signaling and messaging to carry out these operations between the mobile communication devices may be facilitated by a Consent Server, which also maintains a record of the transactions.

A record of each Consent Period and the corresponding {Consent Duration[x]} values is stored with the Sexual Consent Agreement, for example at a Consent Server, disclosed with reference to Step 116. In one embodiment the amount of time the Consent Period can be extended, and/or the number of times it can be extended is capped. For example, the Consent Period may be extended up to two hours from the end of the original {Consent Duration}. In another example, alone or in combination with the previous example, the Consent Period may be extended up to three times, {Consent Duration[1]}, { Consent Duration[2]}, and { Consent Duration[3]}.

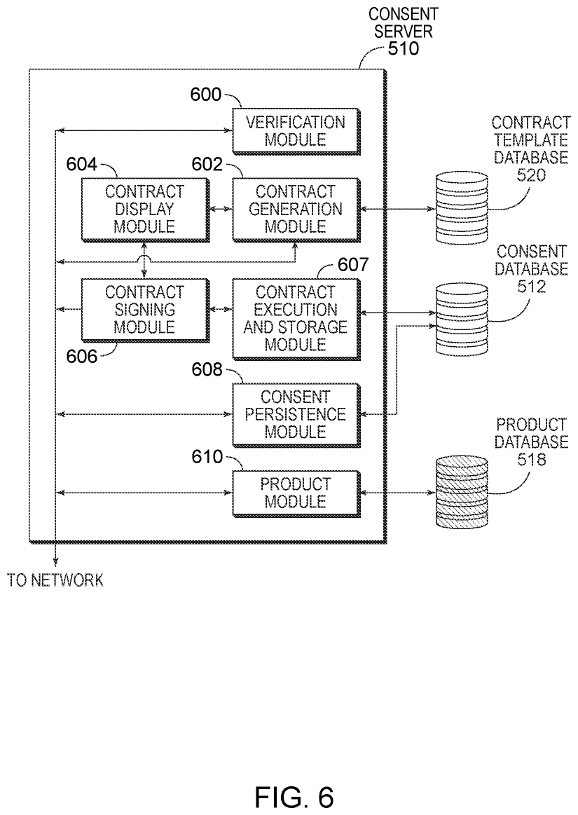

In one embodiment, during the Consent Period, the location of the consenters 502(1) and 502(2), which is available from their mobile communication devices, is periodically determined and stored with the fully executed consent contract. As will be disclosed later with reference to FIGS. 5 and 6, this data is received by a consent server and stored with the fully executed consent contract in a consent database. In this way, a record is made of the history of the locations and movements of the consenters during the Consent Period. This geolocation can provide useful evidence if consent is withdrawn and the circumstances during the Consent Period of disputed.

Consent Persistence and Consent Withdrawal

At step 120, it is determined if consent remained persistent for a period of time following the storing 116 of the fully executed sexual consent contract (the “Consent Persistence Period”). During this time either or both Person 1 or Person 2, may electronically withdraw their consent. Withdrawal of consent may be indicated at any time during the Persistence Period.

The Persistence Period is equal to or greater than the Consent Period. Consent may therefore be withdrawn before, during, or indicated even after any sexual activity or encounter that was previously consented to, as long as it is done within or by the end of the Persistence Period. In one example, the Persistence Period is equal to the Consent Period. In another example, the Persistence Period is equal to the Consent Period plus a number of hours, for example 24 hours. In yet another example, the Persistence is equal to the Consent Period plus a variable or dynamically chosen amount of time that is a function of the ages of the consenters. For example, older people may have a shorter Persistence Period than younger people.

In some cases when engaging in what was intended to be and agreed upon consensual sexual activity, a consenter may want to or need to withdraw their consent. They may, however, be unable to in the moment, because of coercion, assault, escalation of activity in excess of the a priori agreed upon or intended level, the use of unwanted or unasked for force, bullying, intimidation, violence or threats of violence, peer pressure, and the like. Similarly, when drugs or alcohol are involved, either voluntarily or involuntarily such as through the use of “date rape” drugs or the plying of alcohol, the person may become incapacitated after consenting. In these and similar circumstances, it is only after the encounter that the person is able to, or even becomes aware of, or can only upon reflection identify or come to the conclusion that the encounter, though initially intended to be consensual, was in fact or in their opinion non-consensual. Accordingly, in one embodiment, the Persistence Period is greater than the Consent Period to allow one or both consenters sufficient or generous time to electronically report the withdrawal of consent after any encounter.

Consent persistence is determined at step 120. In one embodiment the person may press a button displayed by the app on the display of their mobile communication device indicating that they withdraw or withdrew their consent. In another embodiment, at step 120, the app and/or by the Consent Server (to be disclosed in greater detail) via text message or other electronic communication, proactively queries both consenters as to whether consent was withdrawn for any reason. In various embodiment, these queries occur, alone and in combination, during the Consent Period, around the time of expiration of the Consent Period, between the expiration of the Consent Period and the expiration of the Persistence Period, and upon expiration of the Persistence Period.

In one method, after the expiration of the Consent Period and before the expiration of the Persistence Period, an electronic message about persistent consent is sent to the first mobile communication device and the second mobile communication device. The electronic message asks if consent was persistent. Exemplary messages include “Was your encounter and did your encounter remain consensual?”, “Did you withdraw your consent at any time during your encounter?”, “Were you sexually assaulted at any time during your encounter?”. In one embodiment the electronic message may be sent to the mobile communication devices by the consent server and displayed on each mobile communication device via the app. In another embodiment, the electronic message comprises a text message. The text message is sent from the consent server to each mobile communication device and displayed on each mobile communication device as SMS, MMS, RCS, or equivalent type of message.

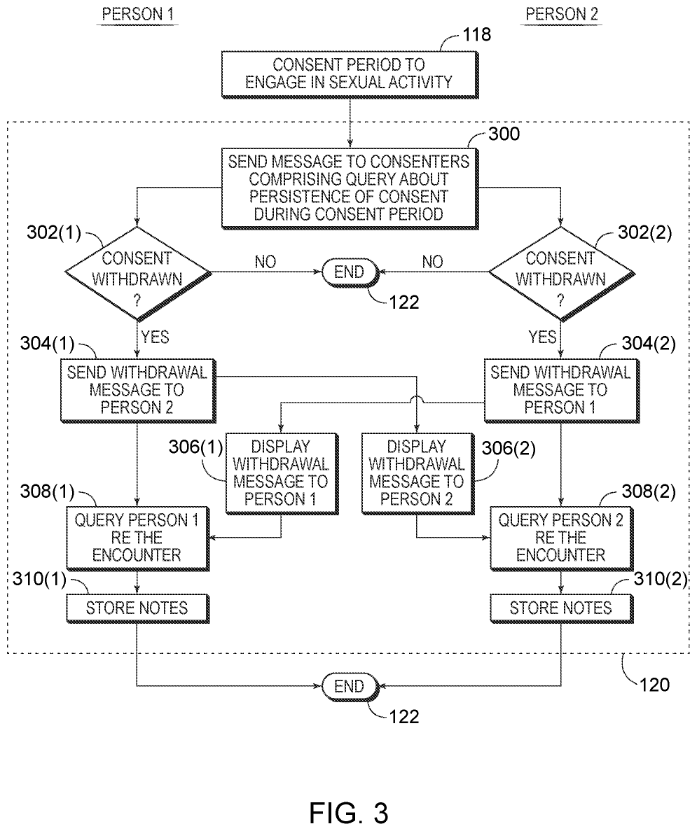

After the electronic message is sent, the method monitors for a response from the first mobile communication device and/or the second mobile communication device. For example, the consent server may monitor for a response. Responses, whether by text message or by way of the app, are recorded, that is they are stored with the fully executed sexual consent contract. A consent persistence method as shown in FIG. 3 will disclose this in greater detail.

More than one electronic message may be sent between the expiration of the Consent Period and the expiration of the Persistence Period. In some cases, even if a response from both parties indicate that consent was persistent, that is not withdrawn at any point, throughout the encounter, one or both parties might change their mind. In other cases, one or both parties may not respond to the electronic message. This may be for a variety of reasons, such as the encounter was in-fact consensual and neither party cares to report that fact, a party is not yet able to emotionally summon the energy report an alleged assault, a party is still incapacitated in some way, a party is not sure whether the entirety of the encounter was consensual and need more time to think about it, and so forth. In any case, additional electronic messages may be sent up until the expiration of the Persistence Period.

If in response to the electronic message, one person indicates that consent did not remain persistent (e.g. it was withdrawn or not possible), an electronic message is sent to the other person. The electronic message may be via the app or a text message. The message may be sent and coordinated by the consent server. Namely, if consent did not remain persistent by the first person, an electronic message is sent to the second mobile communication device. The message indicates that first person withdrew consent. Similarly, if consent did not remain persistent by the second person, an electronic message is sent to the first mobile communication device.

Both parties are queried. In an example where Person 1 indicates that consent was not persistent, an exemplary message sent to Person 2's mobile communication device is “{Name of Person 1} indicated that they withdrew consent or that your encounter was not entirely consensual. Do you agree? Please type your reasons and recollection of the encounter below.” And an electronic message is sent to or displayed on the first mobile communication device, for example “You indicated that you withdrew consent or that your encounter was not entirely consensual. Please type your reasons and recollection of the encounter below.” Both parties are queried. The responses comprising their notes may be sent by text message or by way of the app. These electronic notes are received from the first and second mobile communication devices and are stored with the fully executed sexual consent contract. In one embodiment the notes comprise a video or audio recording.

In the case where a party indicates that consent was withdrawn prior to the expiration of the Consent Period, such as by way of a “Panic Button” on the app, the other party is alerted of the withdrawal of consent, and both parties are still queried to provide notes on the encounter. However, the use of Panic Button is indicative of a potentially dangerous situation. The person who pressed the Panic Button may not be able to enter notes about the encounter immediately. Alerting the other person, may escalate and create a more dangerous situation. So, in one embodiment the parties are queried at the end of the Persistence Period, presumably when they are no longer together. In another embodiment, they are queried only after they are far apart, for example miles apart. Their relative distances can be determined by way of GPS. In another embodiment, the withdrawing person is queried by way of electronic messages even after the Persistence Period has expired to give them sufficient time to document the encounter in safety. In another embodiment the withdrawing person is periodically queried until they provide some explanation of the encounter. In another embodiment the accused person is only queried after the withdrawing person has entered their notes and they have been received and stored. In this way, the method ensures that the withdrawing person is in safety, far away from their alleged attacker. At the same time, the alleged attacker has a fair opportunity to enter notes regarding their recollection of events. In these embodiments, the notes comprising the recollection of the encounter are never shared with the other party. Only the fact that the party withdrew their consent or considered the encounter to be non-consensual is communicated. However, in another embodiment, some or all of the notes are shared to allow a more direct response to allegations. In both of these cases, the parties do not communicate with each other. Their notes are stored with the fully executed sexual consent contract, to be adjudicated later.

FIG. 3 shows the consent persistence method 120 of FIG. 1. The method determines if consent was withdraw. If consent is withdrawn by one person, the other person is electronically notified on their mobile device. They are notified of the fact of withdrawal only. None of the reasons for the withdrawal are shared. Then, both consenters are queried regarding the details of the encounter, their recollections about what occurred, and so forth. Those notes are then stored securely with the fully executed sexual consent contract (step 116), for example, at the Consent Server.

At step 300, an electronic message is sent to the consenters (that is the people who signed the sexual consent contract, {Consenter 1} and {Consenter 2}, which are Person 1 and Person 2 in FIG. 3). The message comprises a query about the persistence of consent during any encounter that occurred during the Consent Period. The electronic message is displayed on the mobile devices of the consenters. Examples of electronic messages comprising queries were given above.

Steps 302(1) and 302(2) monitor for a response to the message sent in step 300. If a response is not received or an affirmative no response is received from Person 1, that is Person 1 affirms in response to the message in step 300 that consent was not withdrawn at any time during the Consent Period, then the process ends at step 122. If an affirmative yes response is received from Person 1, that is Person 1 affirms in response to the message in step 300 that consent was withdrawn (or equivalently, not possible) at any time during the Consent Period, then the process continues to step 304(1).

Similarly, if a response is not received or an affirmative no response is received from Person 2, that is Person 2 affirms in response to the message in step 300 that consent was not withdrawn at any time during the Consent Period, then the process ends at step 122. If an affirmative yes response is received from Person 2, that is Person 2 affirms in response to the message in step 300 that consent was withdrawn (or equivalently, not possible) at any time during the Consent Period, then the process continues to step 304(2).

At step 304(1), which follows if Person 1 indicates that consent was withdrawn in step 302(1), a withdrawal message is sent to the second mobile communication device of Person 2. At step 306(2) the withdrawal message is displayed on the display of the second mobile communication device.

Similarly, at step 304(2), which follows if Person 2 indicates that consent was withdrawn in step 302(2), a withdrawal message is sent to the first mobile communication device of Person 1. At step 306(1) the withdrawal message is displayed on the display of the first mobile communication device.

At step 308(1), Person 1 is queried on the display of their first mobile communication device about the encounter during the Consent Period of step 118. Exemplary queries were disclosed above. As can be seen in the figure, this step follows if either Person 1 or Person 2 indicated by way of their mobile communication device that they withdrew consent. At step 310(1), notes are recorded about the encounter. The notes are entered by Person 1 on their first mobile communication device. This was disclosed above. The method ends at step 122.

Similarly, at step 308(2), Person 2 is queried on the display of their second mobile communication device about the encounter during the Consent period of step 118. Exemplary queries were disclosed above. As can be seen in the figure, this step follows if either Person 2 or Person 1 indicated by way of their mobile communication device that they withdrew consent. At step 310(2), notes are recorded about the encounter. The notes are entered by Person 2 on their second mobile communication device. This was disclosed above. The method ends at step 122.

While the above disclosed written or typed notes on each person's mobile communication device, the notes may also include audio recordings, video recordings, photos, drawings drawn on the screen of the mobile communication device, or any combination thereof. Those notes, as previously disclosed, are stored at the consent server.

Additional Verification

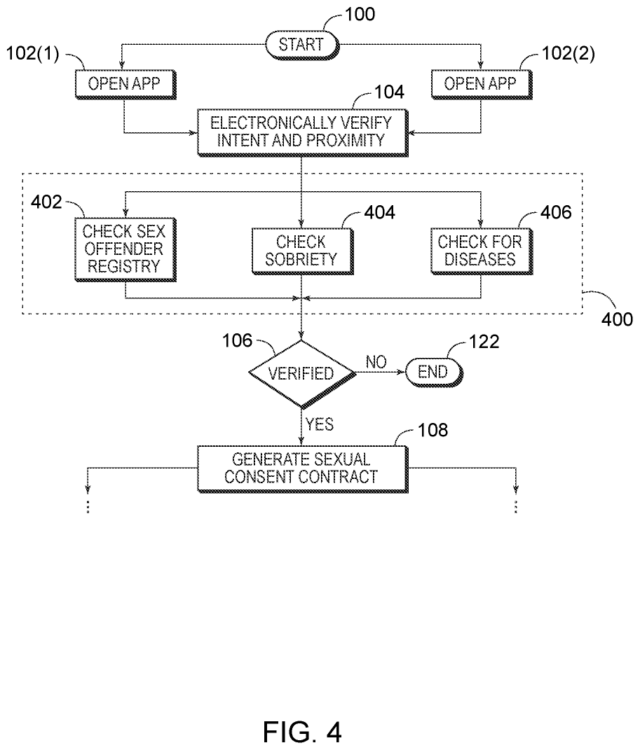

FIG. 4 shows an alternative embodiment of FIG. 1 which includes various optional safety checks. The safety checks verify certain attributes of the users, that is Person 1 and/or Person 2. Depending on the result of any particular safety check, verification may fail at step 106, and the process will end at step 122. The safety checks include, check sex offender register 402, check sobriety 404, and check for diseases 406.

For example, at step 402, a sex offender registry is queried to determine if Person 1 or Person 2 are registered sex offenders. If one of them are on the registry, a message may be displayed on the display of the mobile communication devices, verification may fail, and the method ends 122. One example of a sex offender registry is the “National Sex Offender Public Website” from the United States department of Justice which can be accessed and queried at https://www.nsopw.gov/Home. A search on this registry searches the sex offender registries for all 50 states, The District of Columbia, U.S. territories, and Indian Country. There are states registries that can be individually searched.

The names or other identifying information of the parties may be automatically obtained during the step of electronically verifying 104. In one embodiment, this identifying information may automatically obtained by the app from the user's mobile communication device, which stores the contact information of the owner. The consent server then queries the sex offender database(s) and retrieves the results of the searches.

Automatedly querying databases over a network from a computer such as a server is well known by those having ordinary skill in the art.

In another type of check is a health records check, such as a check to determine if Person 1 or Person 2 have any communicable diseases 406. In one embodiment, the present invention interfaces with contact tracing apps that implement Apple-Google COVID-19 contact tracing and similar technologies (https://www.google.com/covid19/exposurenotifications/) which is hereby incorporated by reference). States such as New York, New Jersey, Pennsylvania, Delaware, and other states are implementing this technology. States are maintaining servers that provide for digital contract tracing. A national server may be put into practice. These servers trace individuals who may have been exposed to or have COVID-19 by way of randomized numerical identifiers, or tokens, stored on each user's mobile communication device, so as to provide a means to store COVID-19 infections in a regional or national database while maintaining privacy.

In one embodiment, at step 406 the token is sent to the consent server. The consent server queries the COVID-19 database and reports the result back to the apps. In another embodiment, the app in combination obtains COVID-19 results from an another app on the user's mobile communication device such as https://apps.apple.com/story/id1524690171 and https://www.google.com/covid19/exposurenotifications/select/. The results are sent to the consent server which sends a message to apps alerting the users if either of them are a COVID-19 risk. Verification may or may not proceed, depending on the preferences of the users. In one embodiment, a message is displayed to the users of the COVID-19 risk of being in contact with each other and both users are asked if they would like to proceed. If they do, the method verification is successful, and the method continues.

In another embodiment, the disease check 406 includes querying a health records database(s) to determine if the users have any communicable disease such as sexual transmitted infections (STI) or disease (STD). Due to privacy regulations, such as HIPAA, this may require additional configuration. When installing the app or configuring the app the user may be asked to authorize access to certain records and asked to enter user/login information to retrieve certain records. In another embodiment, the user may take a photo or download the STI/STD results to their mobile communication device. Those test results may be automatedly scanned and parsed by the app on the mobile communication device, or they may be securely sent to the consent server to be parsed for the results. In either case, a list of STIs/STDs and results of tests may be obtained and stored for the users. In another embodiment, the user may simply be queried by the app if they are free from all sexually transmitted infections and those answers may be transmitted to the consent server and optionally stored with the fully executed sexual consent contract (step 116). If in any cases, a user presents with a positive STI/STD, the other user can be alerted.

In another embodiment, a sobriety check 404 is done on the user's mobile communication device in order to determine if they are intoxicated 404. In one embodiment, if a user is intoxicated, verification does not proceed 106. Some people believe, and there is some developing legal precedent that consent is not possible if a person is intoxicated. In another embodiment, a message is displayed to the users that one or both of the users may be intoxicated, that consent may not be possible, and asking them if they would like to proceed anyway. These results are stored with the fully executed sexual consent contract at step 116.

Constricted or dilated pupils, blurred vision, impaired night vision, droopy eyelids and bloodshot eyes can all be considered symptoms of various drugs. In one embodiment, a sobriety check is performed using the camera of the mobile communication device to image the user's pupil and/or iris. Multiple drugs affect pupil size and reaction (https://www.ncbi.nim.nih.gov/pmc/articles/PMC4545665/ which is hereby incorporated by reference). Some drugs cause pupillary miosis, that is the constriction of the pupil of the eye. Some drugs cause pupillary mydriasis, that is dilation of the pupil of the eye. For example, opiates cause pupil constriction. Cocaine and methamphetamine cause pupil constriction.

The consumption of alcohol causes pupil dilation. In one embodiment, a reference image of user's eye is taken by the user with their mobile communication device. The image may be taken when the app is installed, for example during the registration process. The image may alternatively be taken at any time when the user is not intoxicated. The reference image is stored, for example by the app on the user's mobile communication device or it is sent to and stored on the consent server. The image shows the relative areas of the pupil to the iris when the user is not intoxicated. The ambient light present and/or camera exposure when the photo was taken is also measured. Nearly all mobile communication devices comprise a light sensor to measure ambient light. This sensor, for example, is used to automatically adjust the exposure of photos, to automatically vary the brightness of the display depending on viewing conditions, and so forth.

During the sobriety check of step 404, the app informs the user to take a photo of their eye with their mobile communication device. Image recognition may be used to identify the eye and automatically take a photo when the eye is appropriately focused and in the field of view of the camera. The ambient light present and/or camera exposure of the photo is also recorded. Next, this photo is compared to the reference photo, normalizing for different ambient light conditions and/or exposures. In another embodiment, the user does not take a reference image. In this embodiment, the image taken in step 404 is compared to a database that contains images captured before and after alcohol consumption. Comparative analytics are performed to provide a statistical likelihood that user is intoxicated. One method to measure pupil dilation or constriction under the influence alcohol, including an iris database, “IIITD Iris under Alcohol Influence (IUAI) database”, is described at https://biometrics.ese.msu.edu/Publications/Iris/Aroraetal_IrisRecognitionUnderAlcoholIn fluence_PreliminaryStudy_ICB12.pdf, which is hereby incorporated by reference.

In another embodiment, during the sobriety check of step 404, the app displays a video game that requires visual acuity, dexterity, or quick reaction time. The user is scored according to, for example, their reaction time. Their reaction time is compared to a database of average, non-intoxicated scores. If the user's score falls below a threshold, the user is considered intoxicated. The effects of alcohol ingestion on eye movement and cognition are described at https://doi.org/10.1371/journal.pone.0186061, which is hereby incorporated by reference. The findings confirm that moderate doses of alcohol can change the eye movements of young adults. These alterations may evidence the influence of alcohol in cognitive processes, such as flexibility, attention, and planning, which are required during a task that requires searching a maze.

In another embodiment, the mobile communication device is configured to perform a horizontal gaze nystagmus (HGN) test. An HGN test is similar to a field sobriety test that law enforcement is trained to perform. Nystagmus is the medical term used to describe the involuntary jerking of the eyeballs. When someone is intoxicated by alcohol and/or certain drugs, this jerking becomes more pronounced. As such, the horizontal gaze nystagmus test is used by law enforcement agents to evaluate an individual's nystagmus in order to determine if probable cause exists for a drunk-driving arrest. The mobile communication device is configured to perform this an HGN test at step 404.

One commercial product for law enforcement to check for intoxication based on pupil response is HawkEye (https://newaltas.com/hawkeye-drug-recognition-tool/8578/, which is hereby incorporated by reference). A software development kit, Client-Server Eye Scanning App for Identifying Drug Intoxication, by Softeq Development Corp, is https://www.softeq.com/featured_projects/client_server_eye_scanning_mobile_app, which is hereby incorporated by reference.

One more type of check is an age of consent verification. In this embodiment, the ages of Person 1 and Person 2 are checked to ensure that they have reached the age of consent. Ages may be determined in several ways, for example, from their birthday stored on their mobile communication device, from a user profile database in communication with the consent server, from a scan of an ID such as a driver's license or student ID, and by entering a social security number and querying a database. In one embodiment a user profile database is in communication with the consent server 510. A user profile is made when the user installs their app. The profile includes the user's name and birthdate. The profile may include other data such as an address, profile photo(s), reference photos (such as of eyes as disclosed above), and user preferences. In one embodiment, the app asks the user to take a photo or their ID. The ID is sent to the consent server where it is parsed for information. The user's birthdate is determined. In communication with the consent server is an age of consent database comprising a list of the ages of consent in, for example, each state of the United States. Geolocation data of the user is also sent to the consent server. Once the user's age and location are determined, the consent database is queried to determine to if the user's age is equal to or greater than the age of consent in the locality of the user. If it is not, the verification fails and a sexual consent contract is not generated.

In the embodiment of FIG. 4 and the above disclosure, one or more of the safety checks 402, 404, and 406 are performed after the step of electronically verifying intent and proximity 104, and before generating the sexual consent contract 108. However, some or all of the safety checks may be performed at various points of the method of FIG. 1.

System

FIG. 5 shows the system architecture of a sexual consent system. The system comprises a first mobile communication device 504(1) of a first person 502(1), and second mobile communication device 504(2) of a second person 502(2). An app is running on each mobile communication in accordance with the methods disclosed above, with reference to FIGS. 1-4. Specifically, the app comprises computer executable code which is stored in a memory of the mobile communication, and the code when executed on the process of the mobile communication causes the mobile communication to carry out the methods disclosed above and shown in FIGS. 1-4. FIG. 9 show the architecture of an exemplary mobile communication device.

The mobile communication devices 504(1) and 504(2) are in communication with a network 500. The mobile communication devices 504(1), 504(2) are further in wireless communication with the network 500. The network 500 comprise one or more types of networks such as the wireless network of a mobile carrier, for example, LTE, 4G, 5G, and the like, the Internet, WiFi networks, wired networks, and any other type of network capable of sending and receiving data, such as packetized data networks.

A consent server 510 is in communication with the network 500. Various databases are in communication with the consent server 510, including a contract template database 520, a product database 518, and a consent database 512. The server comprises a microprocessor which is in communication with a memory, a storage device such as non-volatile memory or a hard disk drive, and means for communicating with the network 500. Computer executable code is stored on the storage device, which is loaded into the memory. The computer executable code, when executed on the processor, causes the processor to perform the steps of the methods disclosed above and shown in FIGS. 1-4.

FIG. 6 shows the consent server 510. The consent server comprises a verification module 600 for electronically verifying that the first person and the second person would like to engage in sexual activity and are in proximal communication and cooperating with each other. It comprises a contract generation module 602 for electronically generating as sexual consent contract including the first person and the second person. The contract generation module 602 is in communication with the contract template database 520. The contract generation module 602 is in communication with a contract display module 604 for displaying the sexual consent contract on the displays of the mobile communication devices (504 of FIG. 5). The consent server 510 further comprises a contract signing module 606 electronically signing the sexual consent including receiving the electronic signatures from the mobile device 504. A contract execution module and storage module 607 is for creating a fully executed sexual consent contract, and for storing the fully executed sexual consent contract in the consent database 512. The contract execution and storage module 607 is in communication with the contract storage module 606. A consent persistence module 608 is for determining if consent remains persistent for a limited period of time defined by the Persistence Period. The consent persistence module 608 is in communication with the consent database 512 for storing information about any withdrawal of consent, including notes (see FIG. 3 and associated disclosure above). The modules 600, 602, 604, 606, 607, 608, and 610 are in communication with each other and with the network at shown in FIG. 6.

In an alternate embodiment, the consent server also includes a product module 610 in communication with a product database 518. This will be disclosed in greater detail below.

Turning back to FIG. 5, also in communication with network 500 are optional modules including emergency services 516, health records 519, a sex offender registry 514, and online dating apps 522.

The first person 502(1) and second person 502(2) would like to engage in sexual activity with each other. They take out their mobile communication devices 502(1), 502(2) and open their app to begin the verification process disclosed above. The apps of each mobile communication device 504 signal the consent server 510 to generate verification codes for each device. In response to the signal, the verification codes are transmitted to the respective mobile communication devices 504(1), 504(2). The verification codes are stored by the consent server, for example in the memory or storage device.

The first verification code 506(1) is displayed on the display 507(1) of the first mobile communication device 504(1). Similarly, a second verification code 506(2) is displayed on the display 507(2) of the second mobile communication device 504(2). A scan window 508(1) is displayed on the display 507(1), and a scan window 508(2) is displayed on the display 507(2). The scan window 508 displays the image on the front facing camera of each mobile communication device.

Person 1 502(1) aligns the camera of their first mobile communication device 504(1) to be in view of the second verification code 506(2) displayed on the display 507(2) of the second mobile communication device 504(2). The image of the second verification code 506(2) is captured by the first mobile communication device 504(1), which transmits the captured code to the consent server 510.

Similarly, Person 2 502(2) aligns the camera of their second mobile communication device 504(2) to be in view of the first verification code 506(1) displayed on the display 507(1) of the first mobile communication device 504(1). The image of the first verification code 506(1) is captured by the second mobile communication device 504(2), which transmits the captured code to the consent server 510. The order in which the verification codes 506(1) and 506(2) are scanned is immaterial.

In accordance with the methods disclosed above for electronically verifying, the server authenticates that the code captured in the scan window 508(1) by the first mobile communication device 504(1) is the same as the second verification code 506(2) stored at the consent server 510, and that the code captured in the scan window 508(2) by the second mobile communication device 504(2) is the same as the first verification code 506(1) stored at the consent server 510.

If the codes are authenticated, the server generates the sexual consent contract. In one embodiment, various elements and clauses of the contract are assembled from the contract template database 520. This allows for different contracts to be generated having varying scopes. For example, the contract template database may comprise a non-disclosure agreement which is then included in the sexual consent contract when it is electronically generated. It also allows for contracts to be adjusted based on the location of the users 502(1), 502(2).

The contract and its enforceability depends on various factors. One of those factors may include the locality in which the contract is entered into, and the locality in which it may be interpreted and/or litigated. Examples of localities include countries and states within those countries, such as the States of the United States of America, and venues such as Federal and State courts. As it pertains to the United States of America, it may be advisable or necessary to add or change certain language in the contract depend on the State in which the users 502(1) and 502(2) are entering into the agreement. In these cases, and as disclosed above, the geolocation data obtained by the global positioning subsystem of the mobile communication devices, which comprises information that can be used to identify the mobile communication devices' 504 physical locations, is sent to the consent server 510. In generating the sexual consent contract, the consent server 510 queries the contract template database 520 for elements and clauses that are legally recommended or desired in the locality of the users. This helps ensure that the generated sexual consent contract maintains the highest level of enforceability.

The sexual consent contract is then transmitted from the consent server 510 to each mobile communication device 504(1) and 504(2). The contract is displayed on each of the displays 507(1) and 507(2).

Person 1 502(1) electronically signs the contract displayed on their display 507(1), and Person 2 502(2) electronically signs the contract displayed on their display 507(2). A multiplicity of electronic signatures were disclosed above. However, briefly, in one embodiment, Person 1 502(1) holds their finger on or moves their finger across the fingerprint scanner of their mobile communication device 504(1). Person 2 502(2) does the same with their mobile communication device 504(2). The electronic signatures are transmitted to the consent server, and a fully executed sexual consent contract is created at the consent server, which comprises the identifying information of the parties, their signatures, dates, and any other cryptographic or hashing information required to make execute and validate electronic contract. This fully executed sexual consent contract is stored by the consent server 510 in the consent database 512. With reference to FIG. 3 and the associated disclosure above, consent persistence is determined in a variety of ways. For example, the app displays a panic button on the displays 507 of the mobile communication devices 504. If Person 1 502(1) or Person 2 502(2) touches the button on their screen, a panic signal is transmitted from their device to the consent server 510 and is recorded in the consent database 512 with the fully executed consent contract 512. Or, withdrawal of consent is indicated by way of messages sent by the consent persistence module 608 of the server to the devices 504. Messages are received by the consent persistence module 608 from the devices 504, such as notes. A variety of messages, for example text messages or messages to be displayed by the app, are sent from consent persistence module 608 to the mobile devices 504, and received from the mobile device 504 by the consent persistence module 608. This was all disclosed in detail above with reference to FIG. 3 and the disclosure concerning consent persistence and consent withdrawal. Withdrawal messages and notes are stores by the consent persistence module 608 in the consent database 512 with the fully executed sexual consent contract. In this way, an entire history of the agreement and any violations of it is maintained.

With respect to the panic button, the system of FIG. 5 optionally comprises emergency services 516 in communication with the network. In this embodiment, activating the panic button sends a signal to emergency service 516 which includes the location of the user's mobile communication device. Examples of emergency services 516 include, the police, emergency assistance such as 911, a sexual assault hotline, a therapist, a crisis hotline, or a safety service such as Noonlight (https://www.noonlight.com/, which hereby incorporated by reference) that communicates with a dispatcher that can contact the police. When activating emergency services, a chat window may open on the user's mobile device to chat with the dispatcher or police. Any correspondence is stored with the fully executed consent contract in the consent database 512. A voice call may be initiated to speak with the dispatcher or police. All audio of the call is stored in the consent database 512 with the fully executed consent agreement. Similarly, a video call may be initiated with all video stored in the consent database 512. In various embodiments, the panic button automatically alerts the police or other emergency responders to go to the location of the user.

In one embodiment, the system optionally comprises an online dating module 522 in communication with the network. The online dating module 522 represents one or more online dating services and their servers that communicate with dating apps on users' mobile phones. Examples of online dating services and their apps include, but are not limited to, Tinder, Bumble, Match, Hinge, Happn, Coffee Meets Bagel, Grindr, OkCupid, The League, eharmony, and Plenty of Fish.

These online dating services present through an application on a user's mobile phone. Generally, the app displays a series of photos of users, and sometimes other profile information about the user whose photo is shown. Other user's can like or dislike the photo/profile, thereby expressing their interest or disinterest. For example, a user can swipe right on a photo of someone they are interested in, or swipe left on the photo of someone they are not interested in. Many other means of expressing interest are possible, such as thumbs up or thumbs down, a heart or minus icon, and so forth.

If two users mutually like each other, they are matched and can chat with each other through a messaging platform built into the app. In one embodiment, the online dating services 522 and app includes methods disclosed herein for electronically consenting to sexual activity. For example, the app may include a button that appears after a match has been made to carry out the methods disclosed herein. In another example, the systems and methods may automatically detect when the matched pair are close to each other by way GPS, Bluetooth, or other means disclosed herein. The systems and methods may then query the users if they would like to proceed. In these embodiments, the branded online dating app is adapted to carry out the electronic consent methods as disclosed herein on their mobile communication devices 504, and the app comprises code that communicates with the consent server 510. In this way, online dating companies can enhance and differentiate their service, while still providing a branded experience for their users.

Condoms and Other Products

FIG. 7 shows a condom wrapper 700 comprising a machine-readable verification code 702 being scanned by the mobile communication devices 504(1) and 504(2) of a first person 502(1) and a second person 502(2). Briefly, with additional reference to the method of FIG. 1, the step of electronically verifying intent and proximity 104 includes receiving the verification code 702, which is scanned by the mobile communication devices, in the same manner as and having the same effect as the method of electronically verifying the intent of and proximity of the first person and the second person described with reference to FIGS. 1 and 2.

More generally, FIG. 7 shows a contraceptive product in a sexual activity consent system. A sealed wrapper 700 containing a condom 701 (indicated by the dotted circle). The sealed wrapper 700 may be made of a material such as foil or plastic. In one example, the sealed wrapper 700 is approximately 2.5″×2.5″ square. The sealed wrapper 700 may be larger or smaller, and may be other shapes, such as rectangular or round. The sealed wrapper 700 comprises an interior in which the condom 701 is contained, and an exterior surface. The exterior surface has at least two exterior opposed outer surfaces. The exterior surface may comprise branding, designs, and text. One of the exterior opposed outer surfaces may comprise an expiration date, legal warnings, and other manufacturer information.

The sealed wrapper comprises a machine-readable verification code 702. In this example, the machine-readable verification code 702 comprises a 2-D barcode, such as a QR code the exterior surface. The 2-D barcode is an optically scannable code. When the verification code 702 is scanned by two mobile phones 502(1) and 502(2) that are near each other, for example within several inches or feet of each other, and each mobile phone scans the code 702 at around the same time, for example within about a minute of each other, a sexual consent contract is displayed on the display 507(1) and 507(2) of each mobile device 504(1) and 504(2) in accordance with the above disclosure. The contract is electronically executable by a variety of mean which are disclosed herein. The contract is signed by each user on their device. That is, Person 1 502(1) electronically signs the contract on their device 504(1) and Person 2 502(2) electronically signs the contract on their device 504(2).

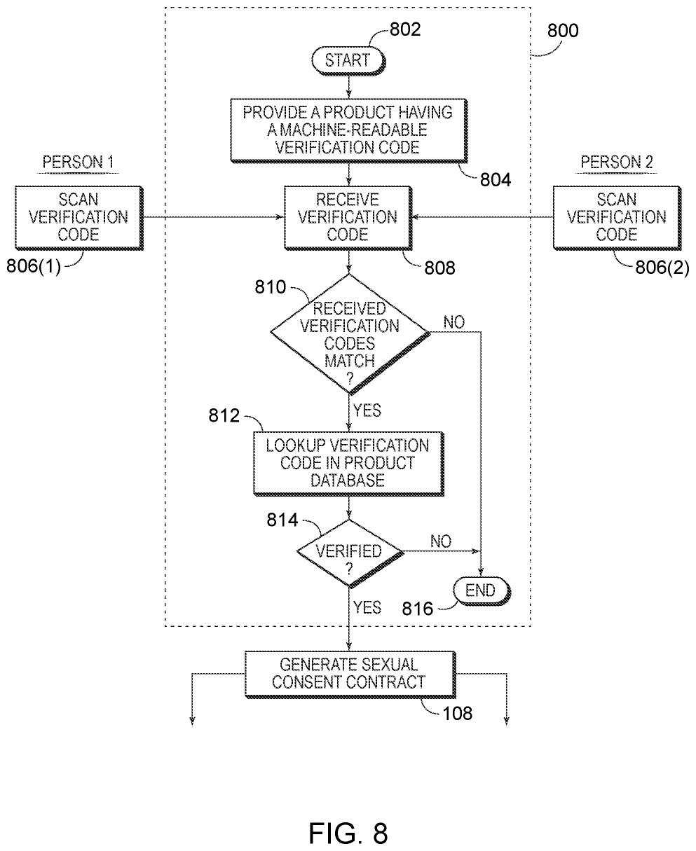

FIG. 8. shows a method of electronically verifying the intent of and proximity of the first person and the second person with a product having a machine-readable verification code. The method 800 begins at step 802. A product is provided having a machine-readable verification code is provided 804.

The product may be any type of product, such as a contraceptive device or packaging for a contraceptive or prophylactic, including a condom, contraceptive gel, birth control pills, and the like. As used herein, the “product” includes apps (application software executed on mobile communication devices) for contraceptive use and monitoring, adapted according to the present invention to display the machine-readable verification code or carry out some or all of the steps of the present methods. One exemplary app available under the tradename NATURAL CYCLES (https://www.naturalcycles.com/ and https://www.fda.gov/news-events/press-announcements/fda-allows-marketing-first-direct-consumer-app-contraceptive-use-prevent-pregnancy, which are hereby incorporated by reference). Another example is a vaginal ring and accompanying app sold under the trademark ANNOVERA.

Types of machine-readable codes comprise an optical barcode, such as a barcode, a two-dimensional barcode (2D code), a matrix barcode like a QR code, and the like. Other types of machine-readable verification codes comprise contactless electronically readable codes such as an electromagnet readable tag, and an electromagnetic readable-writable tag. Examples include, RFID (Radio Frequency Identification) tags, NFC (Near Field Communication) tags, Bluetooth tags, and any other type of tag readable by optical or electromagnetic means, including any type of tag that can be read, scanned, or identified by active or passive means. These types of tags may, for example, be embedded within or on the product. These types of tags may, for example, be printed on the product, applied to or affixed to the product, such as by use of stickers, adhesives, and the like. Tags may also include smart chips or any type of integrated circuit and contactless technology such as those embedded in contactless smart cards like those in bank cards, credit cards, and identification cards. One international standard defining identity cards, contactless integrated circuit cards, or proximity cards used for identification and the protocols for communicating with it, is ISO/IEC 14443 (https://wwwiso.gov/standard/73599.html which is hereby incorporated by reference) and further ICS 35.240.15, identification cards, chip cards, biometrics, including application of cards for banking, trade, telecommunications, transport, etc. (https://wwwiso.gov/ics/35.240.15/x/ which is hereby incorporated by reference).

At step 808, verification codes are received. The codes are scanned by and transmitted from the mobile communication of devices 806(1) and 806(2) of Person 1 and Person 2. FIG. 7 shows Person 1 502(1) and Person 2 502(2) using their mobile communication devices 508(1) and 508(2) to scan machine-readable code 702, in this example a 2-D barcode such as a QR code, on condom 700.

At step 810, the verification codes received in step 808 are compared to determine if they match. If they do not, the verification fails, and the process ends at step 816. If the received verification codes match, then at step 812 a product database is queried for the verification code.

Referring back to FIGS. 6 and 7, the consent server 510 includes a product module 610 in communication with a product database 518. The product database 518 includes a list of valid products adapted to be used with the present invention. By valid products, it is meant, for example, partner companies such as condom and other contraceptive manufacturers. In one, embodiment, unique product codes are generated by or for these partner companies and their products as part of their manufacturing process. These unique product codes are stored in the product database. The machine-readable verification codes, which are included in or on the products, comprise these product codes such that when they are scanned and received at the consent server 510, the consent server 510 is able to identify and verify the product. This allows for validation of products in use or about to be put to use, and tracking of the products and their use. The data may provide useful and marketing and analytics to data to partner companies. This data may be stored along with any ensuing fully executed sexual consent contract in the consent database (512 of FIGS. 5 and 6). In the event of the withdrawal of consent, this data represents potentially valuable evidence in any ensuing sexual assault investigation or litigation.

At step 814, if the lookup is not successful, that is the product does not exist in the product database 518, the verification fails and the process ends 816. Otherwise, the verification is successful, and a sexual consent contract can be generated (step 108) as disclosed herein.

Turning back to FIG. 7, with the above disclosure in mind, a product 700 comprises a machine-readable verification code 702. In this example, the product 700 is a condom and the machine-readable verification code 702 is a 2-D barcode like a QR code. Person 1 and Person 2 scan the machine-readable code 702 with their mobile communication devices 504(1) and 504(2) respectively. In this example, Person 1 positions their device 504(1) close enough to condom 700 so that the camera of their device 504(1) can image the optical code 702. This is illustrated by the diverging dotted lines 708(1) emanating from device 504(1). Similarly, Person 2 positions their device 504(2) close enough to condom 700 so that the camera of their device 504(2) can image the optical code 702. This is illustrated by the diverging dotted lines 708(2) emanating from device 504(2). In an example where the machine-readable code 702 is not optically readable, but rather is a radio tag like a contactless integrated circuit disclosed above, the devices are brought close enough to the product 700 to read the tag.

A scan window 508(1) displayed on the screen 507(1) of device 504(1) shows an image viewed through the camera so that Person 1 can properly align, and the app can capture the verification code. Similarly, a scan window 508(2) displayed on the screen 507(2) of device 504(2) shows an image viewed through the camera so that Person 1 can properly align, and the app can capture the verification code. In the case of a radio tag, the displays 507(1) and 507(2) may display a message such as, “Move your phone closer to the product to scan it” and other instructions to help direct the user to successfully scan the tag.

When the verification code 702 is scanned, each device 504(1) and 504(2) transmits the captured code to the consent server 510, and the various processes which execute on the various elements of the consent server ensue, all of which was disclosed above.

With respect to FIG. 8, the users may scan 806(1) and 806(2) in various ways. In one embodiment, the user opens their app and proceeds through the methods as disclosed above, which includes capturing the optically scannable verification code 702.

In another embodiment, the user opens the camera on their phone to image the barcode 702 directly from the native camera application on their phone, or from a barcode scanning application. Most phones can read barcodes directly from the phone and/or most phones include an application for reading barcodes. In one embodiment, the machine-readable code of the provided product (804 of FIG. 8) is encoded to open the app described in present invention to execute the methods herein. In another embodiment, the machine-readable code is encoded to direct the mobile device to open a website hosted on the consent server, or any other internet accessible resources which can be hosted on the consent server. The website accesses resources that carry out the steps of the methods disclosed herein. In this way the consenters, by way of their mobile devices, can consent to engage in sexual activity and withdraw that consent as disclosed herein, without having to install an application on their phone. In another embodiment, the machine-readable code launches a web application on a user's phone to carry out the methods of the present invention. A web application (“web app” or) is application software that runs on a web server.

In another embodiment, the machine-readable code direct the user's device to run an “instant app” that installs on the user's device, if it is not already on the device. The instant app loads the portions of the app needed to perform the current action. In this way, the user can instantly access the present invention. Google Play Instant is an example of an instant app (https://developer.android.com/topic/google-play-instant/overview, which is hereby incorporated by reference). Similarly, App Clips are mini application that can begin running on an Apple device such as an iPhone without having to be installed (https://developer.apple.com/app-clips/ which is hereby incorporated by reference).

Various other embodiments of the methods and systems disclosed herein are possible. For example, at least some of the methods may be executed in a web browser. For example, verification, displaying the sexual contract, and/or signing the sexual contract may be done by computer executable code running in an interne browser or equivalent. Some methods or elements of methods may be carried out by other forms of electronic communication, such as email, text messages, or a messaging service (for example, WhatsApp, Facebook Messenger, and the like). In one example, verification codes may be sent and received by text message or another form of electronic communication between the consent server and the mobile communication device(s).

The sexual consent contract may be displayed, and an electronic signature received by way of text message or another form of electronic communication. The verification code may include a question such as, “Are you in face-to-face communication with your potential partner?” Various radio-based techniques implemented on a mobile communication device, such as disclosed above, may be used to verify that Person 1 and Person 2 are proximal to each other. Also, the verification may comprise a CAPTCHA or other type of challenge-response test.

One mobile communication device may be used instead of two. In one embodiment the sexual consent contract is displayed and the electronic signatures received on one of the mobile communication devices. In one example of this, a first person initiates the consent method on the first mobile communication device, a second verification code is sent to second user on the second mobile communication device, that second verification code is communicated to the first user and is entered into the first mobile communication device. A first verification code may also be sent to the first mobile communication device. That first verification code is communicated to the second user who text it to consent server. Then the sexual consent contract is displayed on the first mobile communication device. The first and second electronic signatures are received on the first mobile communication. The sexual consent contract may also be sent to the second mobile communication device for review prior to receiving the electronic signatures. Alternatively, the sexual consent contract may be sent to the second mobile communication and the second electronic signature received from that device, while the first electronic signature is received from the first electronic communication device. In view of the present disclosure, many other combinations and permutations are possible.

FIG. 9 shows the architecture of an exemplary mobile communication device. The Wireless Communication Subsystem(s) of many mobile communication devices comprises short range wireless connectivity technology such as Near Field Communication (NFC). In one embodiment, the verifying method of FIG. 2 utilizes NFC at least in part for the generating the verification codes 200(1) and 200(2), communicating the codes 201(1) and 202(2), and receiving the verification codes 202(1) and 202(2). In this embodiment, the mobile communication devices of the consenters are brought within centimeters of each other during the step of electronically verifying intent and proximity (104 of FIG. 1).

The methods and systems disclosed herein may be implemented on any computer communicating over any network. For example, the computers may include desktop computers, tablets, handheld devices, laptops and mobile devices. The mobile devices may comprise many different types of mobile devices such as cell phones, smart phones, PDAs, portable computers, tablets, and any other type of mobile device operable to transmit and receive electronic messages.

The computer network(s) may include the interne and wireless networks such as a mobile phone network. Any reference to a “computer” is understood to include one or more computers operable to communicate with each other. Computers and devices comprise any type of computer capable of storing computer executable code and executing the computer executable code on a microprocessor, and communicating with the communication network(s). For example, a computer may be a web server.

The systems and methods may be implemented on an Intel or Intel compatible based computer running a version of the Linux operating system or running a version of Microsoft Windows, Apple OS, Android, iOS, and other operating systems. Computing devices based on non-Intel processors, such as ARM devices may be used. Various functions of any server, mobile device or, generally, computer may be implemented in hardware and/or in software, including in one or more signal processing and/or application specific integrated circuits.

The computers and, equivalently, mobile devices may include any and all components of a computer such as storage like memory and magnetic storage, interfaces like network interfaces, and microprocessors. For example, a computer comprises some of all of the following: a processor in communication with a memory interface (which may be included as part of the processor package) and in communication with a peripheral interface (which may also be included as part of the processor package); the memory interface is in communication via one or more buses with a memory (which may be included, in whole or in part, as part of the processor package; the peripheral interface is in communication via one or more buses with an input/output (I/O) subsystem; the I/O subsystem may include, for example, a graphic processor or subsystem in communication with a display such as an LCD display, a touch screen controller in communication with a touch sensitive flat screen display (for example, having one or more display components such as LEDs and LCDs including sub-types of LCDS such as IPS, AMOLED, S-IPS, FFS, and any other type of LCD; the I/O subsystem may include other controllers for other I/O devices such as a keyboard; the peripheral interface may be in communication with either directly or by way of the I/O subsystem with a storage controller in communication with a storage device such a hard drive, non-volatile memory, magnetic storage, optical storage, magneto-optical storage, and any other storage device capable of storing data; the peripheral interface may also be in communication via one or more buses with one or more of a location processor such as a GPS and/or radio triangulation system, a magnetometer, a motion sensor, a light sensor, a proximity sensor, a camera system, fingerprint sensor, wireless communication subsystem(s), and audio subsystems.

A non-transitory computer readable medium, such as the memory and/or the storage device(s) includes/stores computer executable code which when executed by the processor of the computer causes the computer to perform a series of steps, processes, or functions. The computer executable code may include, but is not limited to, operating system instructions, communication instruction, GUI (graphical user interface) instructions, sensor processing instructions, phone instructions, electronic messaging instructions, web browsing instructions, media processing instructions, GPS or navigation instructions, camera instructions, magnetometer instructions, calibration instructions, an social networking instructions.

An application programming interface (API) permits the systems and methods to operate with other software platforms such as Salesforce CRM, Google Apps, Facebook, Twitter, Instagram, social networking sites, desktop and server software, web applications, mobile applications, and the like. For example, an interactive messaging system could interface with CRM software and GOOGLE calendar.

A computer program product may include a non-transitory computer readable medium comprising computer readable code which when executed on the computer causes the computer to perform the methods described herein. Databases may comprise any conventional database such as an Oracle database or an SQL database. Multiple databases may be physically separate, logically separate, or combinations thereof.

The features described can be implemented in any digital electronic circuitry, with a combination of digital and analog electronic circuitry, in computer hardware, firmware, software, or in combinations thereof. The features can be implemented in a computer program product tangibly embodied in an information carrier (such as a hard drive, solid state drive, flash memory, RAM, ROM, and the like), e.g., in a machine-readable storage device or in a propagated signal, for execution by a programmable processor; and method steps can be performed by a programmable processor executing a program of instructions to perform functions and methods of the described implementations by operating on input data and generating output(s).

The described features can be implemented in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any type of programming language (e.g., Objective-C, Python, Swift, C#, JavaScript, Rust, Scala, Ruby, GoLang, Kotlin, HTML5, etc.), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. Some elements of a computer are a processor for executing instructions and one or more memories for storing instructions and data. Generally, a computer will also include, or communicate with one or more mass storage devices for storing data files. Exemplary devices include magnetic disks such as internal hard disks and removable disks, magneto-optical disks, and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) for displaying information to the user and a keyboard and a pointing device such as a mouse, trackball, touch pad, or touch screen by which the user can provide input to the computer. The display may be touch sensitive so the user can provide input by touching the screen.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, wired and wireless packetized networks, and the computers and networks forming the Internet.

The foregoing detailed description has discussed only a few of the many forms that this invention can take. It is intended that the foregoing detailed description be understood as an illustration of selected forms that the invention can take and not as a definition of the invention. It is only the claims, including all equivalents, that are intended to define the scope of this invention.

What is claimed is:

1. A method for electronically consenting to engage in a sexual activity between a first person and a second person comprising the steps of:
(a) providing a first mobile communication device of the first person;
(b) providing a second mobile communication device of the second person;
(c) electronically verifying that the first person and the second person would like to engage in sexual activity and are in proximal communication and cooperating with each other;
(d) electronically generating a sexual consent contract for the first person and the second person;
(e) displaying the sexual consent contract on the display of the first mobile communication device and on the display of the second mobile communication device;
electronically signing the sexual consent contract including receiving a first electronic signature of the first person from the first mobile communication device;
(g) electronically signing the sexual consent contract including receiving a second electronic signature of the second person from the second mobile communication device;
(h) creating a fully executed sexual consent contract from the electronically signed sexual consent contract of the first person and the electronically signed sexual consent contract of the second person, wherein the sexual consent contract comprises a consent period during which sexual activity may take place;
(i) storing the fully executed sexual consent contract; and
(j) determining if consent remained persistent for the first person and for the second person during the consent period.

2. The method of claim 1 wherein the step of determining if consent remained persistent comprises the steps of:
(a) sending an electronic message about persistent consent to the first mobile communication device and second mobile communication device, wherein the electronic message is displayed on the display of the first mobile communication device and on the display of the second mobile communication device; and
(b) monitoring for a response to the electronic message from the first mobile communication device or the second mobile communication device.

3. The method of claim 1 wherein if consent did not remain persistent for the first person, sending an electronic message to the second mobile communication device that the first person withdrew consent, and if consent did not remain persistent for the second person, sending an electronic message to the first mobile communication device that the second person withdrew consent.

4. The method of claim 3 further comprising receiving and storing electronic notes about the sexual activity from the first mobile communication device and the second mobile communication.

5. The method of claim 4 wherein the electronics notes comprise at least one of writing, text, drawings, audio, video, and images.

6. The method of claim 1 wherein the step of determining if consent remained persistent comprises the steps of receiving a panic signal from the first mobile communication device or the second mobile communication device, wherein the panic signal indicates a withdrawal of consent.

7. The method of claim 6 further comprising establishing a communication channel between an emergency service and the mobile communication device that transmitted the panic signal.

8. The method of claim 7 further comprising determining a location of the mobile communication device that transmitted the panic signal, and transmitting the location to the emergency service.

9. The method of claim 1 further comprising checking the sobriety of the first person and the second person with the mobile communication devices.

10. The method of claim 1 wherein the step of electronically verifying comprises the steps of:
(a) providing a product having a machine-readable code;
(b) scanning the machine-readable code with the first mobile communication device; and
(c) scanning the machine-readable code with the second mobile communication device.

11. The method of claim 10 wherein the machine-readable code comprises at least one of: an optical code, an electromagnetic tag.

12. The method of claim 10 wherein the product package comprises a contraceptive product.

13. The method of claim 12 wherein the contraceptive product is a condom.

14. The method of claim 1 wherein the first electronic signature and the second electronic signature comprises at least one of: a typed name, typed initials, a checkbox, a fingerprint, initials drawn on the screen of a mobile communication device, signatures drawn on a screen of the mobile communication device, a fingerprint match, a face identification match, an audio recording of a voice, a voice identification match, a video recording, a retinal scan, an eye scan, a biometric identifier.

15. The method of claim 1 further checking if the first person or the second person are sex offenders.

16. The method of claim 1 further comprising checking if the first person or the second person have a communicable disease.

17. The method of claim 1 further comprising checking if the first person and the second person have reached the age of consent.

18. The method of claim 1 wherein the first mobile communication device and the second mobile communication device comprise at least one of: a mobile phone, a tablet, a smart watch, smart glasses.

19. The method of claim 1 wherein the step of electronically verifying comprises the steps of:
(a) generating a first verification code for the first mobile communication device and a second verification code for the second mobile communication device;
(b) communicating the first verification code to the second mobile communication device; and
(c) communicating the second verification code to the first mobile communication device.

20. The method of claim 19,
(a) wherein the first verification code is a first machine-readable optical code, further comprising the step of displaying the first machine-readable optical code on the display of the first mobile communication device; and
(b) wherein the second verification code is a second machine-readable optical code, further comprising the step of displaying the second machine-readable optical code on the display of the second mobile communication device.

21. The method of claim 20,
(a) wherein the step of communicating the first verification comprises the step of scanning the first machine-readable optical code with the second mobile communication device; and
(b) wherein the step of communicating the second verification code comprises the step of scanning the second machine-readable optical code with the first mobile communication device.

22. The method of claim 1 wherein the step of verifying comprises verifying by short-range wireless communication.

23. The method of claim 1 wherein the step of storing further comprises, during the consent period, periodically determining the geolocation of the first and second mobile communication devices, and storing the periodically determined geolocation with the fully executed sexual consent contract.

24. The method of claim 1 further comprising:
(a) providing a legal dispute concerning whether the sexual activity was consensual; and
(b) retrieving the fully executed sexual consent contract as evidence for adjudicating the legal dispute.

25. A system for electronically consenting to engage in a sexual activity between a first person having a first mobile communication device, and a second person having a second mobile communication device, the system comprising:
means for electronically verifying that the first person and the second person would like to engage in sexual activity and are in proximal communication and cooperating with each other;
means for electronically generating a sexual consent contract for the first person and the second person;
means for displaying the sexual consent contract on the display of the first mobile communication device and on the display of the second mobile communication device;
means for electronically signing the sexual consent contract including receiving a first electronic signature of the first person from the first mobile communication device;
means for electronically signing the sexual consent contract including receiving a second electronic signature of the second person from the second mobile communication device;
means for creating a fully executed sexual consent contract from the electronically signed sexual consent contract of the first person and the electronically signed sexual consent contract of the second person, wherein the sexual consent contract comprises a consent period during which sexual activity may take place;
means for storing the fully executed sexual consent contract; and
means for determining if consent remained persistent for the first person and for the second person during the consent period.

26. A computer-readable medium having stored thereon instructions for electronically consenting to engage in a sexual activity between a first person having a first mobile communication device and a second person having a second mobile communication device which, when executed by a processor, causes the processor to perform the steps of:
(a) electronically verifying that the first person and the second person would like to engage in sexual activity and are in proximal communication and cooperating with each other;
(b) electronically generating a sexual consent contract for the first person and the second person;
(c) displaying the sexual consent contract on the display of the first mobile communication device and on the display of the second mobile communication device;
(d) electronically signing the sexual consent contract including receiving a first electronic signature of the first person from the first mobile communication device;
(e) electronically signing the sexual consent contract including receiving a second electronic signature of the second person from the second mobile communication device;
(f) creating a fully executed sexual consent contract from the electronically signed sexual consent contract of the first person and the electronically signed sexual consent contract of the second person, wherein the sexual consent contract comprises a consent period during which sexual activity may take place;
(g) storing the fully executed sexual consent contract; and
(h) determining if consent remained persistent for the first person and for the second person during the consent period.

27. A method for electronically consenting to engage in a sexual activity comprising the steps of:
(a) verifying that a first person and a second person are in face-to-face communication with each other;
(b) displaying a sexual consent contract on the display of at least one of: a first mobile communication device, and a second mobile communication device;
(c) receiving a first electronic signature from the first person and a second electronic signature from the second person, wherein the first and second electronic signatures mean that the first person and the second person agree to terms and condition of the sexual consent contract; and
(d) storing a fully executed sexual consent contract comprising the sexual consent contract in the step of (b) and the electronic signatures in the step of (c), whereby the first person and the second person consent to sexual activity according to the terms and conditions the sexual consent contract.

28. The method of claim 27 further comprising, determining if consent is withdrawn by the first person or the second person.

29. The method of claim 28 further comprising, if consent is withdrawn, receiving information about the withdrawal, and storing the information.

30. The method of claim 28 further comprising, if consent is withdrawn, establishing a communication channel between the person who withdrew consent and an emergency service.

31. The method of claim 27 wherein the step of verifying comprises verifying with a machine-readable code.

32. The method of claim 31 wherein the machine-readable code is at least one of: optically readable, electromagnetically readable.

33. The method of claim 27 wherein the step of verifying comprises verifying with a human-readable code.

34. The method of claim 27 further comprising determining at least one of the following: if the first person or the second person is a sex offender, if the first person or the second person has reached the age of consent, if the first person or the second person is inebriated or intoxicated, if the first person or the second person has a communicable disease.

35. The method of claim 27 further comprising:
(a) providing a legal dispute concerning whether the sexual activity was consensual; and
(b) retrieving the fully executed sexual consent contract as evidence for adjudicating the legal dispute.
